SEQUENCE LISTING (17 sequences)

SEQ ID NO: 1 - DNA, 239 bp, Escherichia coli
gaattcccggtcgggaatttaaaataatcctctattttaagacggcataatacttttttatgccgtttaattcttcgttttgttacctgcctctaactttgtagatctccaaaatatattcacgttgtaaattgtttaacgtcaaatttcccatacagagctaagggataatgcgtagcgttcacgtaactggaggaatgaaatggagtttttcaaaaagacggcacttgcggggatcc

SEQ ID NO: 2 - DNA, 155 bp, Salmonella enterica
gaattctgttttttcctgcattttgttatccatctctaaaaaatactctctgtcggttatatatcgctggttaatcgtatggcgtcacattattcgtctgcaatatagagataatgcgaccagttgacataactggagatataacatggggatcc

SEQ ID NO: 3 - DNA, 23 bp, Artificial sequence
  PCR primer
gcgaattctgttttttcctgcat

SEQ ID NO: 4 - DNA, 24 bp, Artificial sequence
  PCR primer
cggatccccatgttatatctccag

SEQ ID NO: 5 - DNA, 67 bp, Artificial sequence
  PCR primer
gcgaattcaggtaaatttgatgtacatcaaatggtaccccttgctgaatcgttaaggttaggcggta

SEQ ID NO: 6 - DNA, 52 bp, Artificial sequence
  PCR primer
acggatccccatgttatatctccagttatgtcaactaccgcctaaccttaac

SEQ ID NO: 7 - DNA, 29 bp, Artificial sequence
  PCR primer
gacatctagaggtaccaaatgaaaaccgt

SEQ ID NO: 8 - DNA, 28 bp, Artificial sequence
  PCR primer
atatcgcatgccagttcctgcattttac

SEQ ID NO: 9 - DNA, 17 bp, Artificial sequence
  PCR primer
gcctcctgccagcagtg

SEQ ID NO: 10 - DNA, 18 bp, Artificial sequence
  PCR primer
atcggacgatccgcatag

SEQ ID NO: 11 - DNA, 30 bp, Artificial sequence
  PCR primer
atggagctcgcggccggtgatattccgaag

SEQ ID NO: 12 - DNA, 26 bp, Artificial sequence
  PCR primer
ggcgatatctggaatataatttactg

SEQ ID NO: 13 - DNA, 35 bp, Artificial sequence
  PCR primer
gtggaattcgagctcggtcgggaatttaaaataat

SEQ ID NO: 14 - DNA, 38 bp, Artificial sequence
  PCR primer
catcggccgcatgcccgggcctgaaaatctcgccaagc

SEQ ID NO: 15 - DNA, 26 bp, Artificial sequence
  PCR primer
gcggatccaaaatctggattgttggg

SEQ ID NO: 16 - DNA, 18 bp, Artificial sequence
  PCR primer
cggcgtttcacttctgag

SEQ ID NO: 17 - DNA, 17 bp, Artificial sequence
  PCR primer
cccgaaaagtgcacctg

(19) United States
(12) Patent Application Publication
(10) Pub. No.: US 2003/0224009 A1
(43) Pub. Date: Dec. 4, 2003

(54) Bacterial expression systems

(75) Inventors: Tamsin Deborah Terry, Queensland (AU); Michael Paul Jennings, Queensland (AU)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: The University of Queensland

(21) Appl. No.: 10/434,837

(22) Filed: May 9, 2003

Related U.S. Application Data
(63) Continuation of application No. PCT/AU01/01455, filed on Nov. 9, 2001.

(51) Int. Cl. A61K 39/00; A61K 39/38

(52) U.S. Cl. 424/184.1

(57) ABSTRACT

An inducible expression system is provided that includes an inducible ansB promoter co-dependently regulatable by cyclic AMP and anaerobiosis. The expression system is particularly suited to chromosomal expression of immunogenic proteins in attenuated bacterial vaccines. Protein expression from an E. coli-derived ansB promoter is particularly effective in a Salmonella host bacterium.

### FIELD OF TIE INVENTION

THIS INVENTION relates to an inducible expression system suitable for use in vaccines, and methods of immunization, without being limited thereto. More particularly, this invention relates to an expression vector comprising a bacterial ansB promoter isolated from a gene encoding L-asparaginase II, which is suitable for use in vaccines. The expression vector is capable of integration into a bacterial host genome whereby immunogenic proteins may be expressed.

### BACKGROUND OF THE INVENTION

Efficient delivery of vaccines is central to immunization regimes. Attenuated bacteria such as Salmonella have been used for this purpose, both for providing immunization against the attenuated bacteria itself, and for delivery of heterologous proteins useful as immunogens.

In order to deliver heterologous proteins, bacterial expression vectors have been devised in the form of extrachromosomal plasmid vectors and as vectors which are integrated into bacterial chromosomal DNA (Strugnell et al., 1990, Gene 88 57).

Multicopy extrachromosomal plasmid vectors with constitutive promoters tend to provide higher levels of expression, particularly during bacterial propagation, but are inherently unstable and may be lost during propagation.

Chromosomally-integrated vectors have greater stability, but tend to achieve much lower levels of expression than do extrachromosomal vectors.

Therefore, regulatable promoters have been sought which display minimal activity during bacterial growth but increase activity in host tissues (Chatfield et al., 1992, Biotechnology 10 888). Generally, anaerobically-regulated promoters are relatively inactive during bacterial growth where aerobic conditions prevail.

In this regard, the E. coli nirB gene encodes an NADH-dependent nitrite reductase, the nirB promoter becoming active under anaerobic conditions and in conditions of elevated nitrite (Chatfield et al., 1992, supra). The nirB promoter has proven useful in expressing heterologous proteins in Salmonella, from an extrachromosomal plasmid vector. Reference is made, for example, to U.S. Pat. Nos. 5,683,700 and 5,547,664 where a synthetic nirB promoter was used for TetC delivery, although it is noted that the synthetic nirB promoter had the nitrite-responsive element deleted and hence was regulatable by anaerobiosis alone.

In contrast to the nirB promoter, the ansB promoter is positively regulated in a co-dependent fashion by anaerobic conditions and increased cyclic AMP levels. The ansB gene encodes an inducible, secretable L-asparaginase II enzyme (Cedar & Schwartz, 1967, J. Biol. Chem. 242 3753; Cedar & Schwartz, 1968, J. Bacteriol. 96 2043). Both Salmonella and E. coli ansB promoters have been isolated and shown to direct expression of a heterologous protein (β-galactosidase) in bacteria (Jennings et al., 1993a, Mol. Microbiol. 9 155; Jennings et al., 1993b, Mol. Microbiol. 9 165). A subtle difference observed between the E. coli ansB promoter and the Salmonella ansB promoter is that the E. coli promoter requires the presence of both the anaerobically-induced fur gene product (FNR) and the cyclic AMP receptor protein (CRP), whereas the Salmonella ansB promoter is regulated by CRP and an unidentified anaerobically-regulated factor (Jennings et al., 1993b, supra).

The efficacy of either form of the ansB promoter in bacterial expression vectors, such as suitable for vaccines, has not been tested.

### SUMMARY OF THE INVENTION

Therefore, in a first aspect, the present invention resides in an expression vector comprising a promoter co-dependently regulatable by cyclic AMP and anaerobiosis.

The expression vector may be capable of being chromosomally-integrated and maintained in a bacterial cell or may be capable of being maintained extrachromosomally in a bacterial cell.

Preferably, the expression vector is capable of being chromosomally integrated and maintained in a bacterial cell.

In one embodiment, the promoter is co-dependently regulatable by FNR and CRP.

In another embodiment, the promoter is regulated by CRP.

Preferably, the promoter is a regulatory component of an ansB gene.

More preferably, the promoter is a regulatory component of an E. coli or a Salmonella enterica ansB gene.

Even more preferably, the promoter is a regulatory component of an E. coli ansB gene.

In a preferred embodiment, the promoter has a nucleotide sequence selected from the E. coli ansB and S. enterica ansB promoter sequences set forth in FIG. 1.

In a particularly preferred embodiment, the promoter is an E. coli ansB promoter and the bacterial cell is of a Salmonella strain.

In a second aspect, the present invention provides an expression construct comprising an expression vector according to the first-mentioned aspect and a heterologous nucleic acid operably linked to said promoter.

In a third aspect, the invention provides a bacterial cell transformed with an expression construct according to the second-mentioned aspect.

In a fourth aspect, the invention provides a vaccine comprising an expression construct according to the second-mentioned aspect or a transformed bacterial cell according to the third aspect, wherein the heterologous nucleic acid encodes an immunogenic polypeptide.

Preferably, the vaccine includes an immunologically-acceptable carrier, diluent or excipient.

Preferably, the bacterial cell is attenuated.

Preferably, the promoter is an E. coli ansB promoter and the bacterial cell is of a Salmonella strain.

In a fifth aspect, the present invention provides a method of vaccinating a host, said method comprising the steps of administering to said host a vaccine according to the fourth-mentioned aspect.

Suitably, administration of said vaccine induces an immune response by said host.

Preferably, administration of said vaccine protectively immunizes said host.

Also contemplated are expression vectors, expression constructs and vaccines comprising promoter-active fragments, variants and homologs of the aforementioned ansB promoters.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES AND TABLES

TABLE 1: Comparison of relevant biological properties of the E. coli and S. enterica ansB promoters, particularly in terms of regulation by cAMP and anaerobiosis.

TABLE2: Sequences of primers used in construction of ansB expression constructs. Primer sequences are presented 5′ to 3′.

FIG. 1: Alignment of the respective nucleotide sequences of the ansB promoters from E. coli (SEQ ID NO: 1) and S. enterica strain STM1 (SEQ ID NO: 2) cloned into the vectors pMJF1 (pNM481+E. coli ansB promoter) and pNMansB^{S} (pNM481+S. enterica ansB promoter). Restriction sites used for cloning are double-underlined and labelled. The ATG methionine start codons are bolded. The Shine-Dalgarno box is indicated by dotted underlining ( . . . ). The −10 promoter regions are indicated by waved underlining (˜˜˜˜). Asterisks (*) mark identical bases. Ten amino acids (30 bp) of the mature sequence of the E. coli ansB gene are cloned upstream of the lacZ gene in plasmid pMJF1 and are marked with a line above the region of the sequence labeled ansB (residues 203 to 233 of the E. coli sequence). The consensus sequences for the binding sites of the regulatory proteins CRP and FNR are also shown. Upper case letters indicate identical bases whereas lower case letters indicate non-identical bases. This is shown above the E. coli sequence and below the S. enterica sequence. The E. coli ansB promoter and gene sequence may be found at Genbank under accession number M34277; the Salmonella ansB promoter and gene sequence may be found at Genbank under accession number X69868.

FIG. 2: Schematic summary of the use of pCVDaroDansBTetC for construction of a Salmonella strain having a chromosomally-integrated ansB-tetC gene.

FIG. 3: Comparison of heterologous nucleic acid expression driven by chromosomally-integrated or plasmid encoded Samonella enterica ansB promoter (ansB^{S}), E. coli ansB promoter (ansB^{E}) and synthetic nirB promoter (nirB). Expression constructs comprising either the S. enterica ansB promoter, E. coli ansB promoter or nirB promoter upstream of β-galactosidase were inserted into the aroD gene of Salmonella strain STM1/Rif. Plasmids containing the E. coli ansB (pBRansB^{E}), S. enterica ansB (pBRansB^{S}) and nirB (pBRnirB) promoters were introduced into Salmonella strain STM1/Rif. Three separate assays were performed, and the data averaged. All cultures were grown in 15 ml vessels without agitation. Cells were resuspended in sodium phosphate buffer pH=7, sonicated and the lysate clarified. The data have been normalized per μg of total cellular protein.

FIG. 4: In vitro stability of expression constructs in the absence of antibiotic selection. An STM1/Rif colony harbouring each of the plasmids pBRansB^{E}, pBRansB^{S}, pBRnirB and the control plasmid pBR322 was inoculated into LB broth and grown with vigorous aeration for 24 hr. 10 μl of a 1 in 10⁵ dilution of the culture was inoculated into fresh LB and grown for 24 hr. Cells were passaged for five 24 hr periods in total. At each passage, the number of viable organisms and ampicillin resistant organisms in 10 μl of the culture was determined.

FIG. 5: Comparison of expression of fragment C driven by chromosomally-integrated or plasmid encoded E. coli ansB promoter and synthetic nirB promoter. An expression construct consisting of the fragment C gene downstream of the E. coli ansB promoter was inserted into the aroD gene of Salmonella enterica strain STM1/Rif (strain RA07). Plasmid constructs containing the fragment C gene under control of E. coli ansB (plasmid pλansB^{E}-TetC, strain SE01) or nirB (plasmid pλnirB-TetC, strain SE05) were introduced into strain STM1/Rif. Cultures of STM1/Rif, RA07, SE01 and SE05 were grown overnight in 50 ml vessels without agitation. Cells were resuspended in PBS, sonicated and the lysate clarified. Serial 10-fold dilutions were dotted onto a nitrocellulose filter. The filter was blocked overnight with PBS/5% skim milk, incubated for 1 hr with 1:2500 dilution of monoclonal anti-tetanus toxin fragment C antibody (Roche) and washed 3 times with PBS/0.05% Tween 20. The filter was incubated with sheep anti-mouse IgG alkaline phosphatase antibody for 1 hr (1:5000 dilution in PBS/5% skim milk). After incubation the blot was washed 3 times with PBS/0.05% Tween 20, rinsed with alkaline phosphatase buffer and developed using in Nitro Blue Tetrazolium/5-Brom-4-Chloro-3-Indolyl phosphate solution in alkaline phosphatase buffer. As a positive control, purified His-tagged fragment C was serially diluted alongside the samples. Lane 1: STM1/Rif; Lane 2: RA07; Lane 3: SE01; Lane 4: SE05; Lane 5: purified his-tagged fragment C. Dot blotting was used as the monoclonal anti-tetanus toxin fragment C antibody is extremely sensitive to conformational changes, and so works sub-optimally in western blots where proteins have been denatured.

FIG. 6: In vitro stability of expression constructs in the absence of antibiotic selection. An STM1/Rif colony harbouring each of the plasmids pλansB^{E}-TetC (strain SF08), pλnirB-TetC (strain SG03) and the control plasmid pBR322 was inoculated into LB broth and grown with vigorous aeration for 24 hr. 10 μl of a 1 in 10⁵ dilution of the culture was inoculated into fresh LB and grown for 24 hr. Cells were passaged for five 24 hr periods in total. At each passage, the number of viable organisms and ampicillin resistant organisms in 10 μl of the culture was determined.

### DETAILED DESCRIPTION OF THE INVENTION

The present invention is predicated, at least in part, by the present inventors' discovery that the cyclic AMP and anaerobically co-regulated ansB promoter directs considerably higher levels of expression of heterologous polypeptides than does the anaerobically-regulated nirB promoter. This discovery has led the present inventors to create an improved bacterial expression vector and vaccine which may provide enhanced expression of heterologous proteins suitable for immunization. More particularly, the present inventors have discovered that, unexpectedly, the E. coli ansB promoter is actually superior to the S. enterica ansB promoter for the purpose of expressing heterologous polypeptides in Salmonella.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state.

The term “nucleic acid” as used herein designates single-or double-stranded mRNA, RNA, cRNA and DNA, said DNA inclusive of cDNA and genomic DNA.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

By “polypeptide” is also meant “protein”, either term referring to an amino acid polymer.

A “peptide” is a protein having no more than fifty (50) amino acids.

Proteins, polypeptides and peptides may comprise natural and/or non-natural amino acids as are well known in the art.

Nucleic Acids

The vaccines and expression vectors of the present invention may include any promoter which is regulatable by both anaerobiosis and cAMP.

In a preferred embodiment, the promoter is an ansB promoter.

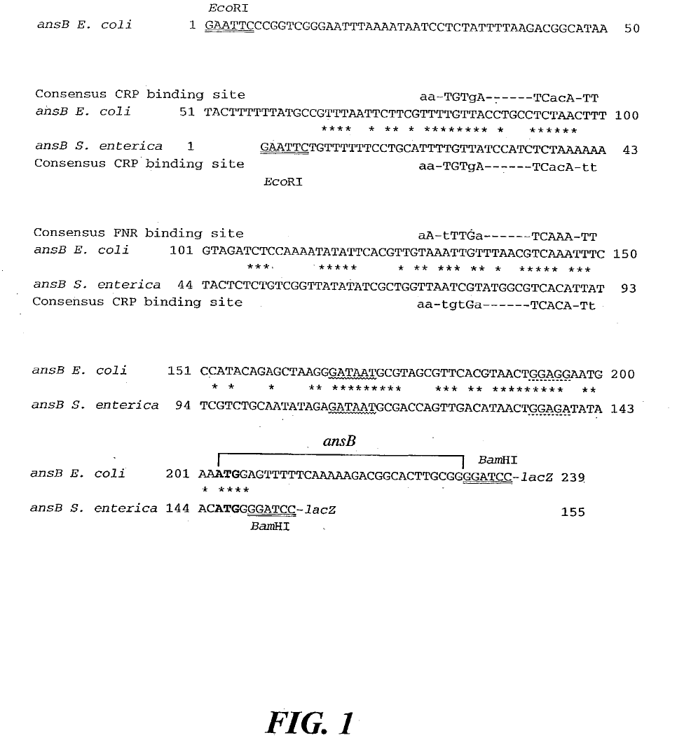

More preferably, the ansB promoter is an E. coli ansB promoter nucleic acid such as set forth in FIG. 1.

The E. coli ansB promoter is set forth in SEQ ID NO: 1.

The S. enterica ansB promoter is set forth in SEQ ID NO: 2.

The invention also contemplates synthetic promoters where FNR and CRP are arranged in a form similar to ansB.

Also contemplated are promoter-active fragments, variants and homologs of ansB promoters useful in vaccines and expression vectors of the invention. These include anaerobically and cAMP-regulated promoters such as ansB promoters from bacterial strains and species other than E. coli and Salmonella, and promoters which regulate expression of genes other than L-asparaginase II.

By “promoter-active fragment” is meant a fragment, portion or segment of an ansB promoter which has at least 1%, preferably at least 5%, more preferably at least 25% or even more preferably at least 50% of the activity of the ansB promoter.

The term “variant” encompasses nucleic acids in which one or more nucleotides have been added or deleted, or replaced with different nucleotides or modified bases (e.g. inosine, methylcytosine). In this regard, it is well understood in the art that certain alterations inclusive of mutations, additions, deletions and substitutions can be made to a reference nucleic acid whereby the altered nucleic acid retains a particular biological function or activity, or perhaps displays an altered but nevertheless useful activity. The term “variant” also include naturally occurring allelic variants.

For example, a promoter set forth in FIG. 1 may be mutated using random mutagenesis, oligonucleotide-mediated (or site-directed) mutagenesis, PCR mutagenesis and cassette mutagenesis. Oligonucleotide-mediated mutagenesis is well known in the art as, for example, described by Adelman et al., 1983, DNA 2:183 using vectors that are either derived from bacteriophage M13, or that contain a single-stranded phage origin of replication as described by Viera et al., 1987, Methods Enzymol. 153 3. Production of single-stranded template is described, for example, in Sections 4.21-4.41 of Sambrook et al. (1989, supra). Alternatively, the single-stranded template may be generated by denaturing double-stranded plasmid (or other DNA) using standard techniques.

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8.4, incorporated herein by reference)

Region-specific mutagenesis and directed mutagenesis using PCR may also be employed to construct promoter variants according to the invention. In this regard, reference may be made, for example, to Ausubel et al., supra, in particular Chapters 8.2A and 8.5, which are incorporated herein by reference.

With regard to random mutagenesis, methods include incorporation of dNTP analogs (Zaccolo et al., 1996, J. Mol. Biol. 255 589) and PCR-based random mutagenesis such as described in Stemmer, 1994, Proc. Natl. Acad. Sci. USA 91 10747 and Shafikhani et al., 1997, Biotechniques 23 304, each of which references is incorporated herein. It is also noted that PCR-based random mutagenesis kits are commercially available such as the Diversify™ kit (Clontech).

In light of the foregoing, the promoters described in FIG. 1 (SEQ ID NO: 1) may include bases encoding ansB protein sequence (ATG beginning at nucleotide 203 of the E. coli sequence) or these coding nucleotides may be excluded.

Also, non-conserved nucleotides 5′ of the E. coli consensus CRP binding site may be deleted while retaining substantial promoter activity.

The skilled person is also referred to Jennings et al., 1993a, supra and Jennings et al., 1993b, supra where the effect of nucleotide mutations and deletions upon ansB promoter activity are investigated.

Promoter-active fragments, variants and homologs of ansB promoters will, ordinarily, display a certain level of sequence identity with a respective sequence set forth in FIG. 1, for example.

Suitably, said fragments, variants and homologs are regulatable by anaerobiosis and cAMP.

In one embodiment, homologs are isolated nucleic acids having at least 60%, preferably at least 70%, more preferably at least 80%, and even more preferably at least 90% sequence identity with a respective promoter sequence set forth in FIG. 1.

Terms used herein to describe sequence relationships between respective nucleic acids include “comparison window”, “sequence identity”, “percentage of sequence identity” and “substantial identity”. Because respective nucleic acids may each comprise (1) only one or more portions of a complete nucleic acid sequence that are shared by the nucleic acids, and (2) one or more portions which are divergent between the nucleic acids, sequence comparisons are typically performed by comparing sequences over a “comparison window” to identify and compare local regions of sequence similarity. A “comparison window” refers to a conceptual segment of typically at least 6 contiguous residues that is compared to a reference sequence, such as used in FASTA comparisons. The comparison window may comprise additions or deletions (i.e., gaps) of about is 20% or less as compared to the reference sequence (which does not comprise additions or deletions) for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference.

A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available 10 from Hitachi Software engineering Co., Ltd., South San Francisco, Calif., USA).

In another embodiment, homologs are isolated nucleic acids which hybridize to a respective promoter sequence set forth in FIG. 1, under at least low stringency conditions, preferably under at least medium stringency conditions and more preferably under high stringency conditions.

“Hybridize and Hybridization” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing between complementary purine and pyrimidine bases, or between modified purines (for example, inosine, methylinosine and methyladenosine) and modified pyrimidines (for example thiouridine and methylcytosine).

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Reference herein to low stringency conditions includes and encompasses:

(i) from at least about 1% v/v to at least about 15% v/v formnamide and from at least about 1 M to at least about 2 M salt for hybridisation at 42° C., and at least about 1 M to at least about 2 M salt for washing at 42° C.; and

(ii) 1% Bovine Serum Albumin (BSA), 1 mM EDTA, 0.5 M NaHPO₄ (pH 7.2), 7% SDS for hybridization at 65° C., and (i) 2× SSC, 0.1% SDS; or (ii) 0.5% BSA, 1 mM EDTA, 40 mM NaHPO₄ (pH 7.2), 5% SDS for washing at room temperature.

Medium stringency conditions include and encompass:

(i) from at least about 16% v/v to at least about 30% v/v formamide and from at least about 0.5 M to at least about 0.9 M salt for hybridisation at 42° C., and at least about 0.5 M to at least about 0.9 M salt for washing at 42° C.; and

(ii) 1% Bovine Serum Albumin (BSA), 1 mM EDTA, 0.5 M NaHPO₄ (pH 7.2), 7% SDS for hybridization at 65° C. and (a) 2× SSC, 0.1% SDS; or (b) 0.5% BSA, 1 mM EDTA, 40 mM NaHPO₄ (pH 7.2), 5% SDS for washing at 42° C.

High stringency conditions include and encompass:

(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;

(ii) 1% BSA, 1 mM EDTA, 0.5 M NaHPO₄ (pH 7.2), 7% SDS for hybridization at 65° C., and (a) 0.1× SSC, 0.1% SDS; or (b) 0.5% BSA, 1 mM EDTA, 40 mM NaHPO₄ (pH 7.2), 1% SDS for washing at a temperature in excess of 65° C. for about one hour; and

(iii) 0.2× SSC, 0.1% SDS for washing at or above 68° C. for about 20 minutes.

In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of Ausubel et al., supra, which are herein incorporated be reference. A skilled addressee will also recognize that various factors can be manipulated to optimize the specificity of the hybridization. Optimization of the stringency of the final washes can serve to ensure a high degree of hybridization.

Typically, complementary nucleotide sequences are identified by blotting techniques that include a step whereby nucleotides are immobilized on a matrix (preferably a synthetic membrane such as nitrocellulose), a hybridization step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20.

According to such methods, Southern blotting involves separating DNA molecules according to size by gel electrophoresis, transferring the size-separated DNA to a synthetic membrane, and hybridizing the membrane bound DNA to a complementary nucleotide sequence.

In dot blotting and slot blotting, DNA samples are directly applied to a synthetic membrane prior to hybridization as above.

An alternative blotting step is used when identifying complementary nucleic acids in a cDNA or genomic DNA library, such as through the process of plaque or colony hybridization. Other typical examples of this procedure is described in Chapters 8-12 of Sambrook et al., supra which are herein incorporated by reference.

Typically, the following general procedure can be used to determine hybridization conditions. Nucleic acids are blotted/transferred to a synthetic membrane, as described above. A wild type nucleotide sequence of the invention is labeled as described above, and the ability of this labeled nucleic acid to hybridize with an immobilized nucleotide sequence analyzed.

A skilled addressee will recognize that a number of factors influence hybridization. The specific activity of radioactively labeled polynucleotide sequence should typically be greater than or equal to about 10⁸ dpm/μg to provide a detectable signal. A radiolabeled nucleotide sequence of specific activity 10⁸ to 10⁹ dpm/μg can detect approximately 0.5 pg of DNA. It is well known in the art that sufficient DNA must be immobilized on the membrane to permit detection. It is desirable to have excess immobilized DNA, usually 10 μg. Adding an inert polymer such as 10% (w/v) dextran sulfate (MW 500,000) or polyethylene glycol 6000 during hybridization can also increase the sensitivity of hybridization (see Ausubel et al., supra at 2.10.10).

To achieve meaningful results from hybridization between a nucleic acid immobilized on a membrane and a labeled nucleic acid, a sufficient amount of the labeled nucleic acid must be hybridized to the immobilized nucleic acid following washing. Washing ensures that the labeled nucleic acid is hybridized only to the immobilized nucleic acid with a desired degree of complementarity to the labeled nucleic acid.

Methods for detecting labeled nucleic acids hybridized to an immobilized nucleic acid are well known to practitioners in the art. Such methods include autoradiography, chemiluminescent, fluorescent and colorimetric detection.

In an embodiment, promoter nucleic acids of the invention may be prepared according to the following procedure:

(i) obtaining a nucleic acid extract from a suitable host;

(ii) creating primers which are optionally degenerate wherein each comprises a respective portion of a nucleotide sequence according to FIG. 1 (SEQ ID NO: 1 or SEQ ID NO: 2); and

(iii) using said primers to amplify, via nucleic acid amplification techniques, one or more amplification products from said nucleic acid extract.

Suitable nucleic acid amplification techniques are well known to the skilled addressee, and include polymerase chain reaction (PCR) and ligase chain reaction (LCR) as for example described in Chapter 15 of Ausubel et al. supra, which is incorporated herein by reference; strand displacement amplification (SDA) as for example described in U.S. Pat. No 5,422,252 which is incorporated herein by reference; rolling circle replication (RCR) as for example described in Liu et al., 1996, J. Am. Chem. Soc. 118 1587 and International application WO 92/01813, and Lizardi et al., (International Application WO 97/19193) which are incorporated herein by reference; nucleic acid sequence-based amplification (NASBA) as for example described by Sooknanan et al.,1994, Biotechniques 17 1077, which is incorporated herein by reference; ligase chain reaction (LCR) as for example described in International Application WO89/09385 which is incorporated by reference herein; and Q-β replicase amplification as for example described by Tyagi et al, 1996, Proc. Natl. Acad. Sci. USA 93 5395, which is incorporated herein by reference.

As used herein, an “amplification product” refers to a nucleic acid product generated by nucleic acid amplification techniques.

By “heterologous nucleic acid” is meant a nucleic acid distinct from an ansB promoter nucleic acid. The heterologous nucleic acid preferably encodes a polypeptide which is to be expressed in bacteria. The polypeptide can be a marker such as LacZ, or more preferably is an immunogenic polypeptide such as TetC. Other immunogenic polypeptides of interest include bacterial antigens such as outer membrane proteins from Haemophilus or Neisseria species, viral proteins such as HIV envelope proteins and parasite antigens.

Expression Vectors and Expression Constructs

Suitably, the heterologous nucleic acid is operably linked to said promoter in said expression vector to form said expression construct.

Expression vectors and constructs of the invention may be self-replicating extra-chromosomal vector such as a plasmid, or a vector that integrates into a bacterial host chromosome.

Preferably, the promoter is an E. coli or Salmonella enterica ansB promoter.

In particular embodiments, the promoter has a nucleotide sequence according to SEQ ID NO: 1 or SEQ ID NO: 2.

In another embodiment, the promoter is homologous to either of SEQ ID NO: 1 or SEQ ID NO: 2

By “operably linked” is meant that said promoter is/are positioned relative to the heterologous nucleic acid to initiate, regulate or otherwise control transcription. Suitably, promoter activity is inducible under the influence of anaerobic conditions and cAMP levels.

In one embodiment, the promoter is a regulatory component of an E. coli ansB gene. It will be appreciated that this promoter is responsive to the FNR and CRP regulatory proteins co-dependently in response to anaerobic conditions and cAMP levels respectively. In another embodiment, the promoter is a regulatory component of an Salmonella ansB gene. It will be appreciated that this promoter is responsive to the CRP regulatory protein in response to cAMP levels, and is also regulated by anaerobiosis through an unknown mechanism.

In this regard, an important distinction between the E. coli and S. enterica ansB promoters is that the S. enterica ansB promoter has two CRP-binding sites (compared to the single site in E. coli where the second regulatory site is an FNR binding site) which function synergistically to direct transcription of a heterologous nucleic acid located downstream (or 3′) of the promoter.

A comparison of the regulatory properties of the E. coli and S. enterica ansB promoters is provided in Table 1.

Expression vectors and constructs of the invention may include one or more other regulatory components in addition to said promoter.

Typically, said one or more regulatory components may include, but are not limited to, nucleotide sequences corresponding to recombination sites, leader or signal sequences, ribosomal binding sites, transcriptional start and termination sequences, translational start and termination sequences, and enhancer or activator sequences.

A preferred recombination sequence is aroD, which facilitates recombination between aroD sequences in the vector and chromosomal aroD sequences.

The expression vector may also include a selectable marker gene to allow the selection of transformed host cells. Selectable marker genes are well known in the art and will vary with the host cell used. Preferred selectable marker genes are bla which confers ampicillin resistance and sacB, which is lethal in the presence of sucrose.

Examples of chromosomally-integratable expression vectors and constructs are:

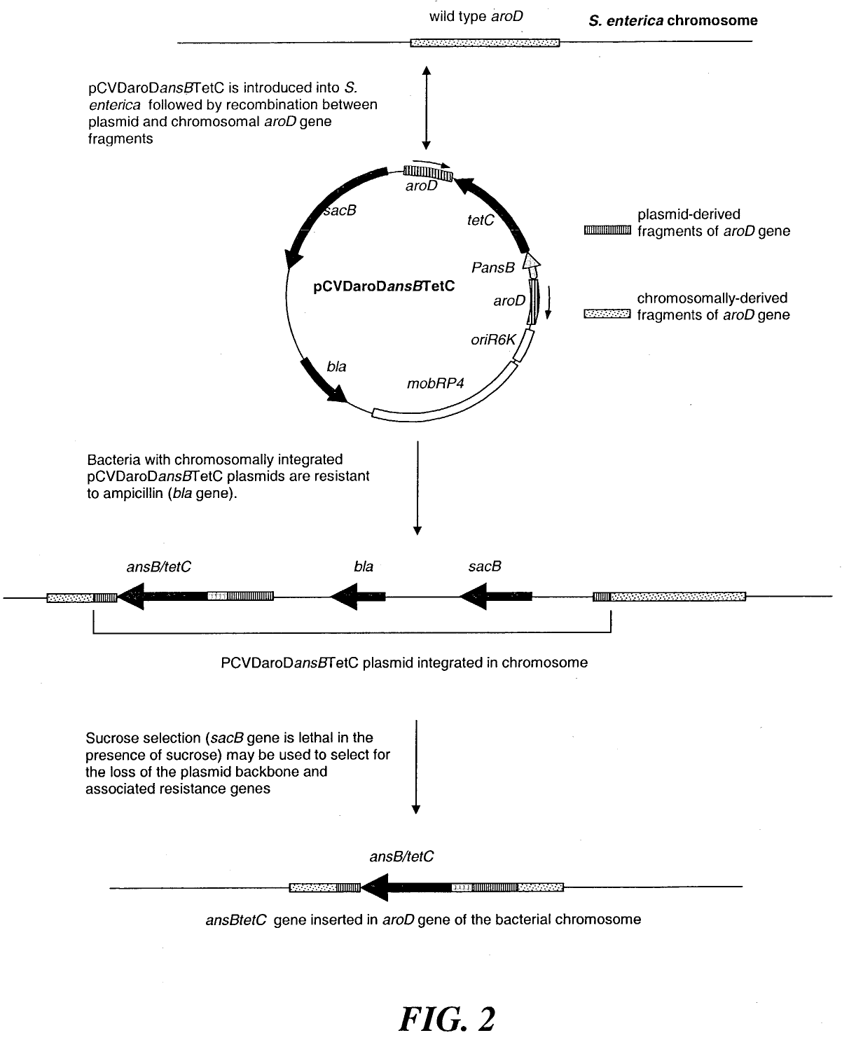

(i) pCVDaroDansBtetC, comprising a heterologous nucleic acid encoding TetC operably linked to the E. coli ansB promoter (see FIG. 2);

(ii) pCVDaroDansB^{S} comprising a heterologous nucleic acid encoding β-galactosidase operably linked to the S. enterica ansB promoter: and

(iii) pCVDaroDansB^{E} comprising a heterologous nucleic acid encoding β-galactosidase operably linked to the E. coli ansB promoter.

Examples of extrachromosomally-maintainable plasmid expression constructs and vectors are:

(i) pBRansB^{E}, which comprises a heterologous nucleic acid encoding β-galactosidase operably linked to the E. coli ansB promoter in a pBR322 plasmid backbone; and

(ii) pBRansB^{S}, which comprises a heterologous nucleic acid encoding β-galactosidase operably linked to the S. enterica ansB promoter in a pBR322 plasmid backbone; and

(iii) pλansB^{E}-tetC, which comprises a a heterologous nucleic acid encoding TetC operably linked to the E. coli ansB promoter.

Vaccines

The present invention provides a vaccine which may be used therapeutically or prophylactically. Accordingly, the invention extends to the production of vaccines containing as actives said heterologous nucleic acid which, preferably, encodes an antigenic or immunogenic polypeptide.

Suitably, the vaccine is administrable to an animal host, preferably an avian or mammal.

By “antigenic” is meant capable of being recognized by components of the host immune system, such as antibodies.

By “immunogenic” is meant capable of eliciting an immune response, preferably a protective immune response upon administration to a host.

Any suitable procedure is contemplated for producing such vaccines. Exemplary procedures include, for example, those described in NEW GENERATION VACCINES (1997, Levine et al., Marcel Dekker, Inc. New York, Basel Hong Kong) which is incorporated herein by reference.

Preferably, the vaccines of the invention are administered in the form of attenuated bacterial vaccines. Suitable attenuated bacteria include Salmonella species, for example Salmonella enterica var. Typhimurium or Salmonella typhi. Alternatively, other enteric pathogens such as Shigella species or E. coli may be used in attenuated form. Attenuated Salmonella strains have been constructed by inactivating genes in the aromatic amino acid biosynthetic pathway (Alderton et al., Avian Diseases 35 435), by introducing mutations into two genes in the aromatic amino acid biosynthetic pathway (such as described in U.S. Pat. No. 5,770,214) or in other genes such as htrA (such as described in U.S. Pat. No. 5,980,907) or in genes encoding outer membrane proteins, such as ompR (such as described in U.S. Pat. No. 5,851,519). The disclosure of each of the aforementioned patents is incorporated herein by reference.

The vaccines of the invention may include an “immunologically-acceptable carrier, diluent or excipient.”

Useful carriers are well known in the art and include for example: thyroglobulin; albumins such as human serum albumin; toxins, toxoids or any mutant crossreactive material (CRM) of the toxin from tetanus, diptheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a T cell epitope of a bacterial toxin, toxoid or CRM may be used. In this regard, reference may be made to U.S. Pat. No 5,785,973 which is incorporated herein by reference

The vaccines of the invention may be administered as multivalent vaccines in combination with antigens of organisms inclusive of the, pathogenic bacteria H. influenzae and other Haemophilus species, M. catarrhalis, N. gonorrhoeae, E. coli, S. pneumoniae, etc.

The “immunologically-acceptable carrier, diluent or excipient” includes within its scope water, bicarbonate buffer, phosphate buffered saline or saline and/or an adjuvant as is well known in the art. Suitable adjuvants include, but are not limited to: surface active substances such as hexadecylanine, octadecylamine, octadecyl amino acid esters, lysolecithin, dimethyldioctadecylammonium bromide, N,N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; lymphokines, QuilA and immune stimulating complexes (ISCOMS).

Multivalent vaccines can be prepared wherein the heterologous nucleic acid encodes:

(i) a plurality of epitopes of an immunogenic polypeptide;

(ii) a plurality of epitopes, each derived from a different immunogenic polypeptide of the same organism; or

(iii) a plurality of epitopes derived from immunogenic polypeptides of different organisms.

In order that the invention may be readily understood and put into practical effect, particular embodiments will now be described by way of the following non-limiting examples.

### EXAMPLE 1

Construction of Plasmid pNMansB^{S} and pNMnirB

Plasmid pMJF1 consists of the promoter probe vector pNM481 (Minton, 1984, Gene 31 269-273) with the E. coli ansB promoter cloned into the BamHI and EcoRI sites of the vector, placing the ansB promoter upstream of β-galactosidase reporter gene (Jennings et al, 1990, J. Bact. 172 149). The plasmids pNMansB^{S} and pNMnirB are identical to pMJF1 except that the E. coli ansB promoter has been replaced with the Salmonella ansB promoter or a synthetic nirB promoter. The ansB promoter from Samonella enterica strain STM1 (Alderton et al. Avian Diseases 35 435-442) was amplified using primers ST01 (5′-GCG AAT TCT GTT TTT TCC TGC AT-3′) and ST02 (5′-CGG ATC CCC ATG TTA TAT CTC CAG-3′). Samples were ampified directly from S. enterica STM1 colonies using an Omn-E Thermal Cycler (Hybaid) with the following program: 95° C. for 2 min then 30 cycles of 94° C. for 30 sec, 52° C. for 30 sec 72° C. for 30 sec and a final extension step of 72° C. for 4 min. After amplification, DNA products were separated on a 2% agarose gel and a 160 bp product corresponding to the ansB promoter was purified from the gel using a Qiagen Qiaquick gel extraction kit.

The synthetic nirB promoter cassette was prepared using primers ST03 (5′-GCG AAT TCA GGT AAA TTT GAT GTA CAT CAA ATG GTA CCC CTT GCT GAA TCG TTA AGG TTA GGC GGT A-3′) and ST04 (5′-ACG GAT CCC CAT GTT ATA TCT CCA GTT ATG TCA ACT ACC GCC TAA CCT TAA C-3′) and DNA polymerase I Klenow fragment (New England Biolabs) to fill in the 3′ recessed ends according to standard protocols (CURRENT PROTOCOLS IN MOLECULAR BIOLOGY, Eds. Ausubel et al., supra).

The DNA fragments were digested with the restriction enzymes EcoRI and BamHI. After digestion, the enzymes were removed by using a Qiagen PCR purification kit. The plasmid pMJF1 was digested with BamHI and EcoRI releasing a ˜300 bp fragment consisting of the E. coli ansB promoter. The digested vector was treated with shrimp alkaline phosphatase, and the vector backbone was separated from the ˜300 bp fragment by agarose gel electrophoresis using a 1% gel. The vector DNA was excised from the gel and purified using a Qiagen Qiaquick gel extraction kit. Vector fragments were ligated to the promoter fragments using T4 DNA ligase, and transformed by electroporation into E. coli DH5α cells. Minipreps from ampicillin resistant transformants were examined by restriction digestion using EcoRI and BamHI to identify plasmids containing the S. enterica ansB promoter and the nirB promoter. The sequences of the respective inserted promoters in representative plasmids (pNMansB^{S} containing the ansB promoter and pNMnirB containing the nirB promoter) were confirmed by DNA sequencing. The S. enterica STM1 sequence was identical to that of S. enterica strain SGSC180 (Genbank Accession X69868).

### EXAMPLE 2

Construction of Plasmid pCVDaroDansB^{S}

The suicide vector pCVD442 (Donnenberg & Kaper, 1991, Infect. Immun. 59 4310-4317, which is incorporated herein by reference) was digested with the blunt-ended restriction enzyme SmaI and the linearised vector was purified by agarose electrophoresis on a 0.8% gel followed by extraction using a Qiagen Qiaex II purification kit. Plasmid pNMansB^{S} was digested with the restriction enzyme AgeI and the enzyme was removed from the DNA solution using a Wizard™ DNA Cleanup Kit (Promega Corp). The DNA was then digested with EcoRI and the enzyme was removed using a Wizard™ DNA Cleanup kit). The resulting fragments were blunt-ended using T4 DNA polymerase as follows: DNA solution in 1× NEB buffer supplemented with 0.1 mg/ml BSA and 125 μM of each of the 4 dideoxynucleotides and 1 μl of T4 DNA polymerase (NEB). The sample was incubated at 37° C. for 5 min before the enzyme was inactivated by heating at 75° C. for 10 min. The blunt-ended DNA fragments were separated on a 0.8% agarose gel and a 4 kb fragment containing the ansB^{S}-lacZ fusion gene was excised and purified using a Qiagen Qiaquick agarose gel extraction kit. The resulting fragment was ligated to the digested pCVD442 suicide vector and electroporated into E. coli DH5αλpir cells. The vector pCVD442 has an R6K origin of replication and is only able to replicate in cells expressing the product of the pir gene. Transformants were plated on LB-Ampicillin plates with X-GAL (5-Bromo-4-chloro-3-indolyl β-D-galactopyranoside) where colonies expressing the lacZ gene and hence producing β-galactosidase were blue. The blue colonies were analysed by restriction digestion to determine the orientation of the ansB^{S}-lacZ cassette in the pCVDans^{B} plasmid.

A fragment of the 5′ end of the aroD gene was amplified by PCR using primer ST06 which incorporates a XbaI site (5′-GAC ATC TAG AGG TAC CAA ATG AAA ACC GT-3′) and primer ST07 which incorporates a SphI site (5′-ATA TCG CAT GCC AGT TCC TGC ATT TTA C-3′) using S. enterica strain 180 galE as a template and using an Onm-E Thermal Cycler (Hybaid) with the following program: 95° C. for 2 min then 28 cycles of 95° C. for 30 s, 50° C. for 30 s, 72° C. for 40 s and a final extension step of 72° C. for 2 min. PCR products were analysed on a 1% agarose gel and the 521 bp fragment of the aroD gene was extracted using a Qiagen Qiaquick gel extraction kit. The PCR product was digested with XbaI and SphI and the enzymes were removed using a Wizard™ DNA Cleanup kit. The vectors pCVD442 and pCVDansB^{S} were similarly digested with SphI and XbaI, treated with shrimp alkaline phosphatase and applied to a 0.8% agarose gel. The linearised vectors were extracted from the gel using a Qiagen Qiaquick gel extraction kit and ligated to the aroD PCR product. Ligations were transformed by electroporation into E. coli DH5αλpir cells. The resulting transformants were plasmids pCVDaroD and plasmid pCVDaroDansB^{S}.

### EXAMPLE 3

Construction of Plasmids pCVDaroDansB^{E} and pCVDaroDnirB

Blunt-ended fragments consisting of the ansB^{E}-lacZ cassette from plasmid pMJF1 and nirB-lacZ cassette were prepared by sequential digestion of the plasmids with AgeI and EcoRI followed by treatment with T4 DNA polymerase to produce blunt ends as described in the construction of plasmid pCVDaroDansB^{S}. The blunt-ended fragment was ligated to plasmid pCVDaroD that had been linearised by digestion with SmaI and gel-purified. Ligations were transformed by electroporation into E. coli DH5αλpir cells and transformants plasmids containing pCVDaroDansB^{E} and pCVDaroDnirB were selected by plating on LB-Ampicillin plates+X-GAL.

### EXAMPLE 4

Development of Rifampicin Resistant Salmonella enterica Strain STM1

A colony of S. enterica strain STM1 was inoculated into 20 ml of LB broth and grown at 37° C. with agitation for 9 hr. Serial dilutions from the culture were plated on LB agar+rifampicin (50 μg/ml) and grown overnight at 37° C. Rifampicin-resistant colonies were subcultured onto LB-rifampicin plates and after a b 2^{nd} overnight growth the pure cultures of STM1/Rif were stored at −70° C. with 20% glycerol. LPS profiles of the strains were checked using standard techniques to confirm that smooth LPS was still expressed by STM1/Rif (Apicella et al., 1994, Meth. Enzymol. 235 242-252).

### EXAMPLE 5

Construction of STM1/Rif with Chromosomally Integrated pCVDaroDansB^{E}, pCVDaroDansB^{S} and pCVDaroDnirB

In order to construct bacterial strains in which plasmids are integrated in the STM1/Rif chromosome, plasmids pCVDaroDansB^{E}, pCVDaroDnirB and pCVDaroDansB^{S} were transformed into the E. coli conjugative donor strain SM10λpir by electroporation. Cultures of SM10λpir cells harbouring each of the two plasmids were cross-streaked onto an LB agar plate with a S. enterica STM1/Rif culture. Plates were incubated at 37° C. for 8 h before the cultures were harvested into LB broth and serial dilutions of each conjugation plated on LB agar plates containing Ampicillin (50 μg/ml) and rifampicin (50 mg/ml) and grown overnight at 37° C. Transconjugants were screened for β-galactosidase activity and the presence of the promoter-lacZ construct on the STM1/Rif chromosome was confirmed by PCR using primers ST08 (5′-GCC TCC TGC CAG CAG TG-3′) and ST09 (5′-ATC GGA CGA TCC GCA TAG-3′).

### EXAMPLE 6

Expression of β-galactosidase in STM1/Rif In Vitro

A single colony of STM1/Rif or derivatives with either plasmid pCVDaroDansB^{E}, pCVDaroDnirB or pCVDaroDansB^{S} integrated into the chromosome was inoculated into 3 ml of LB broth supplemented where appropriate with 100 μg/ml Ampicillin. The bacteria were grown for 8 hr at 37° C. with vigorous shaking. Cultures were diluted 1:1000 into 15 ml of LB broth (with ampicillin where appropriate) in 15 ml screw-capped tubes. The bacteria were grown ovenight at 37° C. without shaking to mid-log phase. Cultures were harvested by centrifugation and washed once and resuspended in phosphate buffer (1 A₆₀₀ nm/ml). Cells were lysed by sonication followed by centrifugation to remove the cell debris. The sonicates were assayed for β-galactosidase activity (Miller J. H., 1972 Assay of β-galactosidase. In Experiments in Molecular Genetics, Cold Spring Harbor Laboratory Press, Cold Spring Harbour, N.Y.). Sonicates (10 μl or 100 μl) were incubated with 900 ml of Z buffer (60 mM Na₂PO₄, 40 mM NaH₂PO₄, 10 mM KCl, 1 mM MgSO₄ and 40 mM β-mercaptoethanol. Tubes were preheated at 28° C. and the reaction initiated by the addition of 0.2 ml of 4 mg/ml ONPG (o-nitrophenyl-β-D-galactoside; Sigma-Aldrich). Reactions were incubated for 15 min and stopped by the addition of 0.5 ml of 1 M Na₂CO₃ before measuring the absorbance at 420 nm. The protein concentration in the sonicates was determined using a BCA Protein Assay (Pierce). The β-galactosidase specific activity was expressed as units/tg protein where a unit is defined as (1000×A_{420 nm})/[(time in min)×(volume of culture used)].

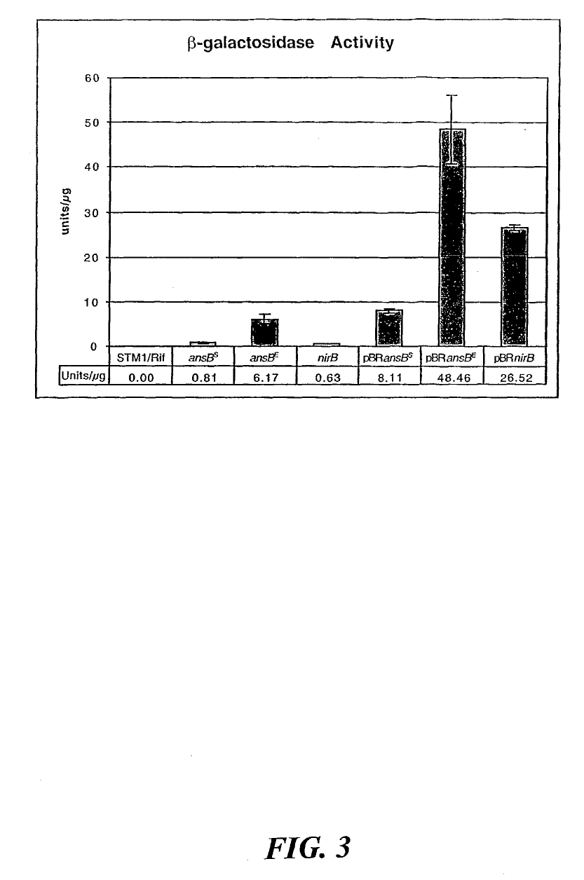

Referring to FIG. 3, the β-galactosidase assays showed that the chromosomally-integrated E. coli ansB promoter displayed at least 4 times, and up to 10 times, more expression that the S. enterica ansB promoter or the nirB promoter. Thus, somewhat unexpectedly, the E. coli ansB promoter diplayed more activity in at least the particular Salmonella strain tested, than did the corresponding Salmonella ansB promoter.

### EXAMPLE 7

Comparison of Expression Levels of β-galactosidase from ansB Promoters Encoded on Muliticopy Plasmids or Chromosomally

Plasmid pBR322 has been used extensively for extrachromosomal heterologous antigen delivery systems in Samonella. The promoter-lacZ cassettes from plasmids pMJF1, pNMnirB and pNMansB^{S} were subcloned into pBR322 as follows: pBR322 was digested with AvaI, followed by heat inactivation of the enzyme. The ends of the linearised vector were blunt-ended using T4 DNA polymerase and the DNA purified using a Wizard™ DNA Cleanup Kit. The DNA was then digested with EcoRI and the digestion products were separated on a 1% agarose gel. A 2.9 kbp fragment incorporating the plasmid origin and ampicillin cassette was isolated using a Qiagen Qiaquick gel extraction kit. The plasmids pMJF1 and pNMansB^{S} were digested with AgeI followed by heat inactivation of the enzyme. The linearised vector was blunt-ended using T4 DNA polymerase, purified using a Wizard™ DNA Cleanup kit and further digested with EcoRI. A ˜4 kb band encoding the promoter-lacZ fragment was gel isolated and ligated to the digested pBR322 vector before electroporation into E. coli DH5α cells resulting in colonies harbouring plasmids pBRansB^{E} and pBRansB^{S}. These plasmids were transformed by electroporation into S. enterica strain STM1/Rif and expression levels of β-galactosidase compared to those of STM1/Rif strains with chromosomally integrated promoter-lacZ cassettes.

Referring to FIG. 3, the chromosomally encoded E. coli ansB promoter produced similar levels of β-galactosidase activity to that produced by the Salmonella ansB promoter on a multicopy plasmid. In addition, β-galactosidase activity from the single copy, chromosomally-encoded E. coli ansB promoter is about one quarter of the activity produced by the nirB promoter in a multicopy plasmid (pBRnirB).

### EXAMPLE 8

In Vitro Stability of Plasmids pBRansB^{S}, pBRansB^{E} and pBRnirB

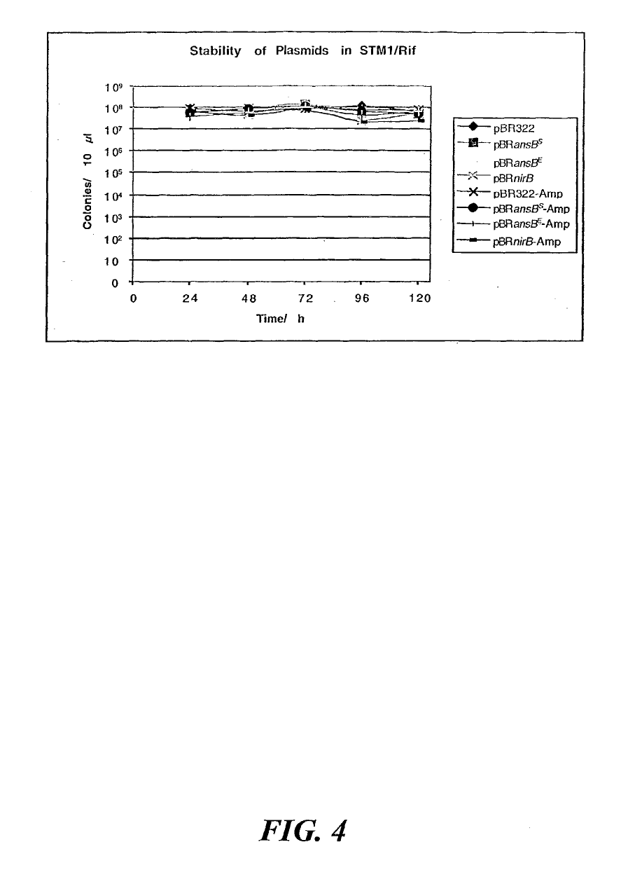

Colonies of STM1/Rif harbouring plasmids pBRansB^{E}, pBRnirB, pBRansB^{S} or pBR322 from a freshly-grown plate were inoculated into 10 ml of LB broth in a 50 ml screw cap tube and grown for 24 hr with vigorous shaking. Bacteria were subcultured into LB broth for five 24 hr periods. At each subculture, serial dilutions of 10 μl aliquots were plated onto parallel LB plates with or without ampicillin. At the end of the final subculture colonies from the LB-ampicillin plates were replica plated onto LB-ampicillin plates containing X-GAL and the resulting colonies were scored for the expression of β-galactosidase. After 5 culture periods of 24 h each in the absence of antibiotics, almost identical numbers of each culture were isolated on both LB and LB-ampicillin plates (FIG. 4) indicating that all 4 plasmids were stable in vitro under these conditions. All the colonies tested on LB-ampicillin X-gal plates harbouring plasmids pBRansB^{E}, pBRansB^{S} or pBRnirB expressed β-galactosidase.

### EXAMPLE 9

Analysis of Intracellular β-galactosidase Expression

Cultures of STM1/Rif with chromosomally integrated pCVDaroDansB^{E} or pCVDaroDansB^{S} are grown to an A₆₀₀ nm of 0.5. Bacterial cells are harvested and resuspended in pre-warmed tissue culture media. S. enterica strains are then added to monolayers of a macrophage-like cell line at a multiplicity of infection of ten bacteria per host cell and incubated for 1 hr to allow phagocytosis to occur. Infected monolayers are washed three times with either PBS or tissue culture media to remove non-adherent bacteria. Extracellular bacteria are killed by, a 1 hr incubation with fresh medium supplemented with 100 μg/ml gentamycin. Cell layers are washed twice and incubated in fresh medium with 20 μg/ml gentamycin. At various times after infection the monolayers are harvested by washing twice and then lysed with PBS containing 0.5% v/v Triton X-100. (Marshall et al., 2000, Vaccine 18 1298). Alternatively, cells may be lysed using sterile, distilled water and vigorous pipetting (Everest et al., 1995, FEMS Microbiology Lett. 126 97). Lysates are then diluted and plated to determine the number of intracellular bacteria. β-galactosidase activities are determined for each lysate.

### EXAMPLE 10

Construction of pCVDaroDansBTetC, pλansB-TetC and pλnirB-TetC

A schematic description of plasmid pCVDaroDansBTetC is shown in FIG. 2. Plasmid pCVDaroDins was constructed by amplifying ˜500 bp from the C-terminus of the S. enterica strain STM1 aroD gene by PCR using primers ST10 and ST11 that introduce SacI and EcoRV restriction sites respectively at either end of the PCR product. Digestion of the PCR product with SacI and EcoRV and plasmid pCVDaroD with SacI and SmaI followed by ligation and transformation results in the construction of plasmid pCVDaroDins that includes two sections of the aroD gene separated by restriction sites. Antigens and promoters can be cloned into these sites either by restriction digestion of purified plasmids or by digestion of PCR products synthesised with appropriately designed primers followed by ligation to digested pCVDaroDins vector.

For example, the nucleic acid (tetC) encoding the C terminal fragment (“fragment C” or “TetC”) of the toxin from Clostridium tetanii was amplified from plasmid pKK/pagC/C frag (Dunstan et al., 1999, Infect. Immun. 67 5133-5141) using primers ST24 (includes a BamHI site) and ST25 (binds downstream of a HindIII site in plasmid pKK/pagC/C frag). The PCR fragment was digested with BamHI and HindIII and ligated to BamHI/HindIII digested pQE-32 plasmid DNA (Qiagen). Expression of fragment C from the resulting plasmid, pQE32-TetC was confirmed by transferring the plasmid to the E. coli expression strain M15/pREP4 and analysing cell extracts from induced cultures by western blotting using monoclonal anti-Tetanus toxin C fragment antibody (Roche).

To facilitate cloning of various antigens under the control of the ansB^{E} and nirB promoters vectors pλMJF1, pBRMJF1lacZ, pBnirBlacZ were constructed, all derivatives of plasmid pBR322. Plasmid pλMJF1 was constructed in 2 steps. First, an unrelated gene cloned into pQE-30 (Qiagen) which had sites for the restriction enzymes EcoRI, MotI, PstI and HindIII (junction with pQE32 vector) just after the 3′ end of the gene was amplified by PCR using primers ST15 (includes EagI site) and pQE-Pro. A fragment containing the λt₀ terminator was excised by digestion with EcoRI and EagI, gel-purified and ligated to pBR322 that had previously been digested with EcoRI and EagI resulting in plasmid A. The ansB^{E} promoter was excised from pMJF1 by digestion with HindIII, treatment with T4 DNA polymerase to remove overhangs, and further digestion with EcoRI. The resulting fragment was ligated to plasmid A that had been digested with NotI, and the site converted to a blunt end by T4 polymerase and then the DNA digested with EcoRI. To construct plasmid pBRMJF1lacZ, plasmid PMJF1 (Jennings et al., 1990, supra) was digested with-AgeI, treated with T4 DNA polymerase to ‘blunt-end’ the restriction site, and then digested with BamHI. A 4 kB fragment containing the lacZ gene was gel-isolated and ligated to BamHI/SmaI digested pλMJF1. To construct plasmid pBRnirB-lacZ, the nirB promoter from plasmid pNMnirB was amplified by PCR using primers ST03 and ST04, digested with EcoRI and BamHI, and ligated to EcoRI/BamHI digested pBRMJF1lacZ.

The fragment C gene from plasmid pQE32-TetC was excised using the restriction enzymes BamHI and HindIII, and ligated to pλMJF1 also digested with BamHI and HindIII, resulting in plasmid pλansB^{E}-TetC. To construct plasmid pCVDaroDansBTetC, the ansB-tetC gene cassette from pλansB^{E}-TetC was amplified by PCR using primers ST12 (includes EcoRI and SacI sites) and ST15 (includes SmaI, SphI and EagI sites). The PCR product was cloned into pCVDaroDins following digestion with SacI and SphI.

To construct plasmid pλnirB-TetC, plasmid pBRnirBlacZ was digested with BamHI, ClaI and EagI. A BamHI/EagI fragment consisting of the vector backbone and nirB promoter was gel-isolated and ligated to the tetC gene fragment from plasmid pλMJF1-TetC excised using BamHI and EagI.

### EXAMPLE 11

Construction of STM1/Rif with Chromosonally-Integrated ansB-tetC Gene

Plasmid pCVDaroDansBTetC was transformed into E. coli strain S17.1λpir. The transformed S17.1λpir strains were cross-streaked with S. enterica strain STM1/Rif on LB agar plates for 8 hrs. Transconjugants were selected on LB plates containing 50 μg/ml Ampicillin and rifampicin. Transconjugants with the plasmid integrated into the STM1/Rif chromosome were selected using PCR and assessed for fragment C expression and sensitivity for growth on media containing 5% sucrose.

An alternative method for transforming cells, whereby the plasmids are introduced into an intermediate r⁻ m⁺ S. enterica var. typhimurium strain by electroporation or conjugation and then the single crossover chromosomal DNA introduced into a wild-type STM1 strain by P22 transduction, may also be used (Miller et al., 1989, Mol. Gen. Genet. 215 312).

### EXAMPLE 12

Expression of Tetanus Toxin Fragment C In Vitro

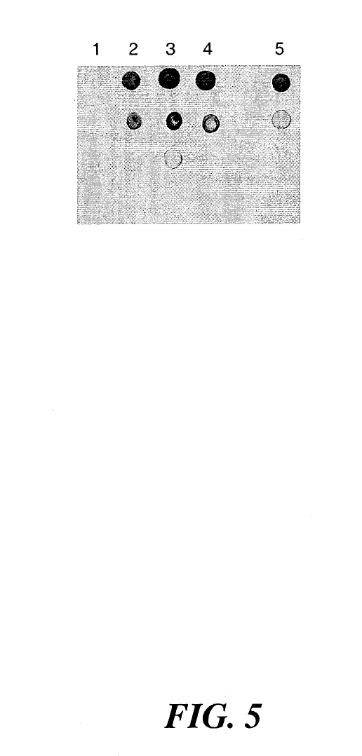

Fragment C expression was examined in STM1/Rif strains harbouring chromosomally integrated pCVDaroDansBTetC, plasmid pλansB^{E}-TetC, and plasmid pλnirB-TetC. A colony of each strain was inoculated into a 50 ml Falcon tube containing 50 ml of LB broth supplemented with Ampicillin (100 μg/ml) where appropriate. Cells were grown overnight at 37° C. without agitation, harvested, washed in PBS and resuspended at an A₆₀₀ nm of 10. Cells were disrupted by sonication and cell debris pelleted by centrifugation at 13,000× g for 5 min. The concentration of total cell protein in the supernatant was determined using a BCA Protein Assay (Pierce) and found to be similar. Serial dilutions of the supernatant were applied to nitrocellulose membrane, and the fragment C protein detected using monoclonal anti-Tetanus toxin C fragment antibody (Roche) and goat anti-mouse IgG alkaline phosphatase conjugated secondary antibody. As a control, purified his-tagged fragment C was also applied to the membrane. Both chromosomal and plasmid encoded fragment C constructs produced significant amounts of TetC protein (FIG. 5).

### EXAMPLE 13

In Vitro Stability of Plasmids pλansB^{E}-TetC and pλnirB-TetC

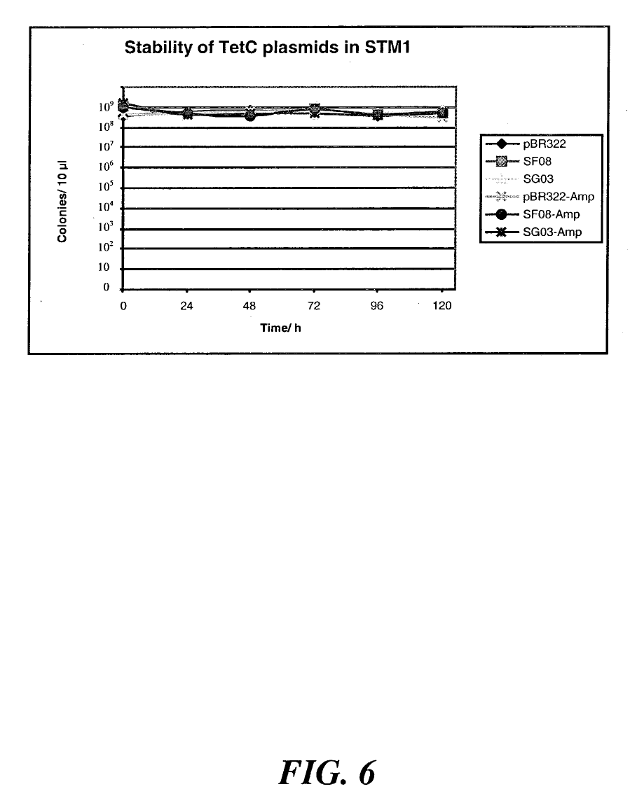

Colonies of STM1/Rif harbouring plasmids pλansB^{E}-TetC an pλnirB-TetC or pBR322 from a freshly-grown plate were inoculated into 10 ml of LB broth in a 50 ml screw cap tube and grown for 24 h with vigorous shaking. Bacteria were subcultured into LB broth for five 24 hr periods. At each subculture, serial dilutions of 10 μl aliquots were plated onto parallel LB plates with or without ampicillin. At the end of the final subculture 50 colonies from each of the LB plates were replica plated onto LB-ampicillin plates. After 5 culture periods of 24 h each in the absence of antibiotics, almost identical numbers of each culture were isolated on both LB and LB-ampicillin plates (FIG. 6) indicating that both plasmids were are stable in vitro under these conditions. All the colonies replica-plated from LB onto LB-ampicillin plates grew.

### EXAMPLE 14

Comparison of the In Vivo Kinetics of STM1/Rif Strains in BALB/c Mice

Groups of Salmonella-negative 6-8 week old BALB/c mice are orally inoculated with bacteria derived from a mid-logarithmic phase culture (for example 0.2 ml of 5×10⁸ cfu/ml in PBS) of Salmonella strains harbouring plasmids or chromosomally inserted β-galactosidase or fragment C genes. At various time points during the first 15 days after inoculation mice are killed and the spleens, Peyers patches and mesenteric lymph nodes removed and homogenized in 5 ml of PBS (as for example described in U.S. Pat. No. 5,683,700). The total number of Salmonella and the number of ampicillin resistant bacteria are determined by plating on LB and LB-Ampicillin plates. In addition the proportion of cells expressing β-galactosidase where the inoculum is a strain incorporating a β-galactosidase gene is determined by plating on LB plates containing X-GAL.

### EXAMPLE 15

Immune Responses in BALB/c Mice Inoculated with STM1/Rif Strains with Chromosomally Encoded Fragment of TetC or β-galactosidase

Groups of 7, 6-8 week old BALB/c mice were orally inoculated with 0.2 ml of 5×10⁹ cfu/ml or intraperitoneally injected with 0.1 ml of 1×10⁸ cfu/ml of bacteria in PBS. Bacterial strains were derived from cultures grown without agitation overnight at 37° C. At day 14, mice were inoculated a second time. Sera was collected and tetanus toxoid-specific or β-galactosidase-specific serum antibody responses were quantified by western blotting.

Serum antibody responses were measured in mice. Purified β-galactosidase was applied to a 6% SDS-PAGE gel, and blotted onto a nitrocellulose filter. The filter was cut into strips, and incubated for 2 hr with a 1:200 dilution of sera from day 0 and day 14 from each mouse. Data will also be obtained at day 28 and day 36. Strips were washed 3 times with PBS/0.05% Tween 20 and incubated for 1 hr with goat anti-mouse IgG alkaline phosphatase conjugated antibody. Strips were then washed 3 times with PBS/0.05% Tween 20, equilibrated with alkaline phosphatase buffer, and developed in Nitro Blue Tetrazolium/5-Brom-4-Chloro-3-Indolyl phosphate detection buffer for 4 h.

At day 14 after inoculation 1 mouse in the group inoculated i.p. with strain TD01 (STM1/Rif with chromosomally incorporated ansB^{E}-LacZ) and 2 mice in the group inoculated orally with strain TE02 (STM1/Rif with pBRansB^{E}) gave responses above background as measured by western blotting.

Serum-antibody responses against fragment C were quantified using a standard end-point ELISA (Dunstan et al., 1999, supra). At day 14 after inoculation 1 mouse in each of the groups that were inoculated i.p. with strain RA07 (STM1/Rif with chromosomally incorporated ansB^{E}-TetC), i.p. or orally with strain SE01 (STM1/Rif with pλansB^{E}-TetC) and orally with strain SE05 (STM1/Rif with pλnirB-TetC) had titres above that of background.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

It will also be appreciated that all patent and scientific literature and computer programs described in this specification are incorporated in their entirety herein by reference.

TABLE 1

| Function | E. coli ansB promoter | S. enterica ansB promoter |
|---|---|---|
| Upstream regulator site | CRP | CRP |
| Downstream regulator site | FNR | CRP |
| Express ion | | |
| 1. Anaerobic culture | High | High |
| 2. Anaerobic + glucose | Low | Low |
| 3. Aerobic | Minimal | Low |
| Optimal expression | Anaerobiosis, low level of | Anaerobiosis, low level of |
| conditions | catabolites (e.g. glucose) | catabolites (e.g. glucose) |
| Anaerobic regulator | FNR | mechanism unknown, no direct |
| | | link with FNR, but strains |
| | | lacking FNR have lowered |
| | | expression levels |
| Catabolite regulator | CRP | CRP |
| | Promoter is co-activated in | Promoter is co-activated by |
| | a co-dependent manner by one | two dimers of CRP (one at |
| | dimer each of CRP and FNR, | each site) which function |
| | binding to their respective | synergistically. |
| | sites | |
| Expression in crp mutant | Expression ˜⅙^{th} of wild- | Expression reduced to ˜10% |
| strain under anaerobic | type | of w.t. Salmonella strain |
| conditions | | |
| Expression in an fnr mutant | Almost non-existent | Used oxrA mutant (homologue |
| strain under anaerobic | | of fnr) of Salmonella. |
| conditions | | Expression reduced to |
| | | slightly more than 50%. |
| | | Mechanism is unknown |

TABLE 2

| Oligonucleotide name | Sequence | |
|---|---|---|
| ST01 | GCGAATTCTGTTTTTTCCTGCAT | (SEQ ID NO:3) |
| ST02 | CGGATCCCCATGTTATATCTCCAG | (SEQ ID NO:4) |
| ST03 | GCGAATTCAGGTAAATTTGATGTACATCAAATGGTACCC | (SEQ ID NO:5) |
| | CTTGCTGAATCGTTAAGGTTAGGCGGTA | |
| ST04 | ACGGATCCCCATGTTATATCTCCAGTTATGTCAACTACC | (SEQ ID NO:6) |
| | GCCTAACCTTAAC | |
| ST6 | GACATCTAGAGGTACCAAATGAAAACCGT | (SEQ ID NO:7) |
| ST7 | ATATCGCATGCCAGTTCCTGCATTTTAC | (SEQ ID NO:8) |
| ST08 | GCCTCCTGCCAGCAGTG | (SEQ ID NO:9) |
| ST09 | ATCGGACGATCCGCATAG | (SEQ ID NO:10) |
| ST10 | ATGGAGCTCGCGGCCGGTGATATTCCGAAG | (SEQ ID NO:11) |
| ST11 | GGCGATATCTGGAATATAATTTACTG | (SEQ ID NO:12) |
| ST12 | GTGGAATTCGAGCTCGGTCGGGAATTTAAAATAAT | (SEQ ID NO:13) |
| ST15 | CATCGGCCGCATGCCCGGGCCTGAAAATCTCGCCAAGC | (SEQ ID NO:14) |
| ST24 | GCGGATCCAAAATCTGGATTGTTGGG | (SEQ ID NO:15) |
| ST25 | CGGCGTTTCACTTCTGAG | (SEQ ID NO:16) |
| PQE-Pro | CCCGAAAAGTGCACCTG | (SEQ ID NO:17) |

1. An expression vector comprising a promoter co-dependently regulatable by cyclic AMP and anaerobiosis, which expression vector is capable of at least one of:
(i) being chromosomally-integrated and maintained in a bacterial cell; and
(ii) being maintained extrachromosomally in a bacterial cell.

2. The expression vector of claim 1, wherein the promoter is co-dependently regulatable by FNR and CRP.

3. The expression vector of claim 1, wherein the promoter is regulatable by CRP.

4. The expression vector of claim 1, wherein the promoter is a regulatory component of a bacterial ansB gene.

5. The expression vector of claim 3, wherein the promoter is a regulatory component of at least one of an E. coli ansB gene and a Salmonella enterica ansB gene.

6. The expression vector of claim 3, wherein the promoter is a regulatory component of an E. coli ansB gene.

7. The expression vector of claim 1, wherein the promoter has a nucleotide sequence selected from the group consisting of SEQ ID NO: 1 and SEQ ID NO: 2.

8. The expression vector of claim 1, comprising a promoter-active fragment of a sequence selected from the group consisting of SEQ ID NO: 1 and SEQ ID NO: 2.

9. The expression vector of claim 7, wherein the promoter is regulatable by anaerobiosis and cAMP and has a nucleotide sequence that hybridizes at least under low stringency conditions with a nucleotide sequence selected from the group consisting of SEQ ID NO: 1 and SEQ ID NO: 2.

10. An expression construct comprising a heterologous nucleic acid and the expression vector of claim 1, wherein the heterologous nucleic acid is operably linked to said promoter.

11. The expression construct of claim 10, wherein the heterologous nucleic acid encodes an immunogenic protein.

12. The expression vector of claim 11, wherein the immunogenic protein is selected from the group consisting of TetC, β-galactosidase, and a respective fragment of TetC and β-galactosidase.

13. The expression construct of claim 10, wherein the promoter has a nucleotide sequence set forth in a sequence selected from the group consisting of SEQ ID NO: 1 and SEQ ID NO: 2.

14. The expression construct of claim 10, comprising a promoter-active fragment of a sequence selected from the group consisting of SEQ ID NO: 1 and SEQ ID NO: 2.

15. The expression construct of claim 10, which is selected from the group consisting of:
(i) pCVDaroDansBtetC;
(ii) pCVDaroDansB^{S};
(iii) pCVDaroDansB^{E};
(iv) pBRansB^{E};
(v) pBRansB^{S}; and
(vi) pλansB^{E}-tetC.

16. The expression construct of claim 10, wherein the promoter is regulatable by anaerobiosis and cAMP and has a nucleotide sequence that hybridizes at least under low stringency conditions with the nucleotide sequence of selected from the group consisting of SEQ ID NO: 1 or SEQ ID NO: 2.

17. A bacterial cell transformed with the expression construct of claim 10.

18. The bacterial cell of claim 17, wherein the promoter is an E. coli ansB promoter and the bacterial cell is of a Salmonella strain.

19. The bacterial cell of claim 17, wherein the ansB promoter is chromosomally-integrated and maintained in the bacterial cell.

20. The bacterial cell of claim 17 which is attenuated.

21. A vaccine comprising the expression construct of claim 10.

22. The vaccine of claim 21 further comprising an immunologically-acceptable carrier, diluent or excipient.

23. A vaccine comprising a transformed bacterial cell according to claim 17.

24. The vaccine of claim 23, wherein the bacterial cell is attenuated.

25. The vaccine of claim 24, wherein the promoter is an E. coli ansB promoter and the bacterial cell is of a Salmonella strain.

26. A method of vaccinating a host, the method comprising the step of administering to the host a vaccine according to claim 21.

27. The method of claim 26 wherein the vaccine induces an immune response by the host to a protein encoded by the heterologous nucleic acid.

28. The method of claim 27, wherein the immune response is selected from the group consisting of an humoral response and a mucosal response.

29. The method of claim 27, wherein administration of the vaccine protectively immunizes the host.
